(19) United States
(12) Patent Application Publication
(10) Pub. No.: US 2009/0234226 A1
(43) Pub. Date: Sep. 17, 2009

(54) CHEMICAL LIQUID INJECTOR

(75) Inventor: Shigeru Nemoto, Tokyo (JP)

Correspondence Address: KNOBBE MARTENS OLSON & BEAR LLP, 2040 MAIN STREET, FOURTEENTH FLOOR, IRVINE, CA, 92614, US

(73) Assignee: Nemoto Kyorindo Co., Ltd., Tokyo (JP)

(21) Appl. No.: 12/295,857

(22) PCT Filed: Apr. 2, 2007
(86) PCT No.: PCT/JP2007/057413

§ 371 Date: Nov. 19, 2008

(30) Foreign Application Priority Data
Apr. 4, 2006 (JP) 2006-103355

(51) Int. Cl. A61M 5/145

(52) U.S. Cl. 600/432

(57) ABSTRACT

The injector (100) comprises a protocol setting means (410) for setting the injection protocols of the contrast medium and the diluent, and a speed control means (440) for controlling the action of the piston drive mechanism (130) in accordance with the injection protocols. In the injection protocols the injection speed (VA) of the contrast medium is higher than the injection speed (VB) of the diluent when a time (T1) elapses after the injection start, the injection speed (VA) of the contrast medium monotonously decreases whereas the injection speed (VB) of the diluent monotonously increases for the period from the time (T1) to a time (T2), and the injection speed (VB) of the diluent is higher than the injection speed (VA) of the contrast medium when the time (T2) elapses.

### TECHNICAL FIELD

The present invention relates to a chemical liquid injector which injects a chemical liquid into a patient, and more particularly, a chemical liquid injector which injects a contrast medium and physiological saline as a chemical liquid into a patient.

### BACKGROUND ART

Presently available medical imaging diagnostic apparatuses include X-ray CT (Computed Tomography) scanners, MRI (Magnetic Resonance Imaging) apparatuses, PET (Positron Emission Tomography) apparatuses, ultrasonic diagnostic apparatuses, angiography apparatuses, CT angiography apparatuses, MRA (MR angiography) apparatuses and the like.

When the abovementioned apparatuses are used, a contrast medium and physiological saline may be injected into a patient in order to provide more favorable diagnostic images. Since the contrast medium is a liquid with consistency, chemical liquid injectors for automatically injecting the chemical liquid have been put into practical use. For example, Patent Document 1 (Japanese Patent Laid-Open No. 2004-194721) has described an apparatus in which a syringe filled with a contrast medium and a syringe filled with physiological saline are mounted and the two syringes are individually operated by a driving mechanism to inject the contrast medium while it is diluted with the physiological saline at an arbitrary dilution ratio.

On the other hand, Patent Document 2 (Japanese Patent Laid-Open No. 2004-113475) has described achievement of optimal contrast in images by injecting a contrast medium at a variable speed over time.

An optimal injection pattern for a contrast medium, however, has not been obviously known depending on body sections to be tested and purposes. For example, when an X-ray angiography apparatus is used to examine the heart of a patient, a contrast medium is distributed in the whole body through bloodstreams but reaches the right atrium and right ventricle at a different point in time from that when it reaches the left atrium and left ventricle. This presents a problem in that it is difficult to examine the atrial septum or ventricular septum with the angiography apparatus.

Patent Document 1: Japanese Patent Laid-Open No. 2004-194721

Patent Document 2: Japanese Patent Laid-Open No. 2004-113475

### DISCLOSURE OF THE INVENTION

The present invention has been made in view of the abovementioned problem and it is an object thereof to provide a chemical liquid injector which can inject a contrast medium in accordance with an optimal injection pattern for a diagnostic image of a particular body section.

The present invention relates to the following.

1. An injector wherein at least two syringes including a syringe filled with a contrast medium and a syringe filled with a diluent are mounted, and comprises at least two piston driving mechanisms which individually operates the two syringes,

the injector further comprising:

a protocol setting means for setting an injection protocol in which each of injection speeds of the contrast medium and the diluent is represented over time; and

a speed control means for controlling operation of the two piston driving mechanisms in accordance with the set injection protocol,

the protocol setting means can receive an setting of an injection protocol (referred to as a cross injection protocol) in which the injection speed of the contrast medium is higher than the injection speed of the diluent from start of injection to time T1, the injection speed of the contrast medium is monotonously reduced and the injection speed of the diluent is monotonously increased from time T1 to time T2, and the injection speed of the diluent exceeds the injection speed of the contrast medium at time T2.

2. The injector according to claim 1, further comprising a protocol storing means storing the cross injection protocol.
3. The injector according to claim 2, wherein the protocol storing means stores a basic pattern of the cross injection protocol, and the basic pattern is read out in response to read-out operation performed by the protocol setting means.
4. The injector according to any one of claims 1 to 3, wherein the injection speed of the contrast medium is constant from the start of injection to time T1 and is linearly reduced from T1 to T2, and

the injection speed of the diluent is constant from the start of injection to time T1 and is linearly increased from T1 to T2.

5. The injector according to claim 4, wherein the injection speed of the diluent is equal to 0 ml/sec from the start of injection to time T1, and

the injection speed of the contrast medium is equal to 0 ml/sec at time T2.

6. The injector according to claim 4 or 5, wherein the sum of the injection speed of the contrast medium and the injection speed of the diluent is constant from the start of injection to time T2.
7. The injector according to any one of claims 1 to 6, wherein the injector is used in X-ray diagnosis of a heart disease.

### EFFECT OF THE INVENTION

According to the present invention, it is possible to provide a chemical liquid injector which can inject a contrast medium in accordance with an optimal injection pattern for a diagnostic image of a particular body section.

Particularly, according to the present invention, it is possible to provide an injector optimal for X-ray diagnosis of heart diseases with an X-ray angiography apparatus.

### BRIEF DESCRIPTION OF THE DRAWINGS

FIG. 1 is a diagram for showing an exemplary injector according to the present invention;

FIG. 2 is a perspective view showing the outer appearance of the exemplary injector;

FIG. 3 is a graph showing a cross injection protocol performed by the injector according to the present invention; and

FIG. 4 is a block diagram for explaining relationships between respective means of the injector.

### DESCRIPTION OF REFERENCE NUMERALS

100 INJECTOR
101 INJECTION CONTROL UNIT
102 CABLE
103 MAIN OPERATION PANEL
104 DISPLAY
107 HAND UNIT
108 CABLE
110 INJECTION HEAD
111 STAND
112 ARM
113 HEAD BODY
114 CONCAVE PORTION
130 PISTON DRIVING MECHANISM
200C, 200P SYRINGE
210 CYLINDER
220 PISTON
230 CONNECTION TUBE
410 PROTOCOL SETTING MEANS
420 INPUTTING MEANS
430 PROTOCOL STORING MEANS
440 SPEED CONTROL MEANS

### BEST MODE FOR CARRYING OUT THE INVENTION

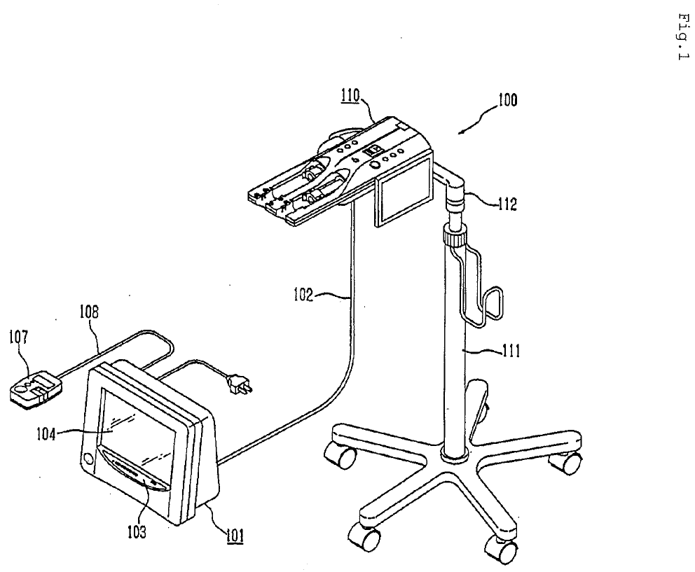

An embodiment of the present invention will hereinafter be described with reference to FIGS. 1 to 4. An imaging diagnostic system in which an injector according to the present invention is used comprises a injector 100 and an X-ray angiography apparatus (not shown) which serves as an imaging diagnostic apparatus. Injector 100 is wire-connected or wirelessly connected to the angiography apparatus.

As shown in FIG. 1, by way of example, injector 100 includes injection head 110 which is attached to an upper portion of arm 112 connected to stand 111 and is connected to injection control unit 101 formed as a separate component through cable 102. Injection control unit 101 has main operation panel 103, display 104, hand unit 107 connected thereto through cable 108 and the like.

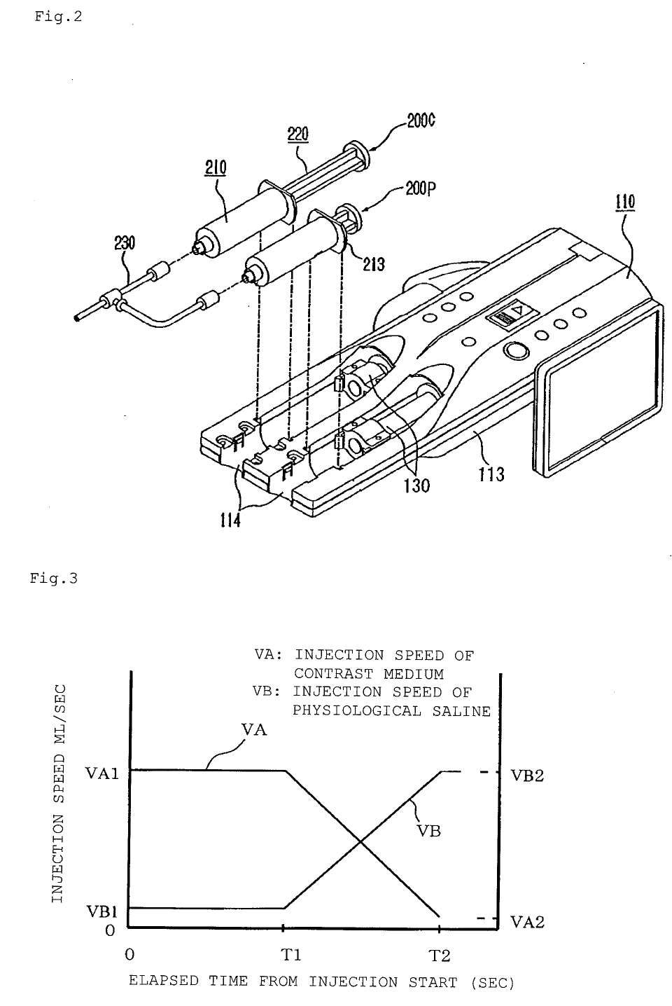

As shown in FIG. 2, head body 113 of injection head 110 has two concave portions 114 as syringe holding mechanisms formed in its upper surface. Two syringes 200C and 200P are mounted in these concave portions 114. Each of syringes 200C and 200P has cylinder 210 and piston 220. Syringe 200C is filled with a contrast medium for angiography, while syringe 200P is filled with physiological saline as a diluent. The ends of the two syringes mounted on head body 113 are connected with connection tube 230. Pistons 220 of the syringes are pushed by piston driving mechanisms 130 movable individually to allow injection of the contrast medium, injection of the physiological saline, and simultaneous injection of both.

The known structure can be used generally as the structure of the piston driving mechanism and the control mechanism or the like.

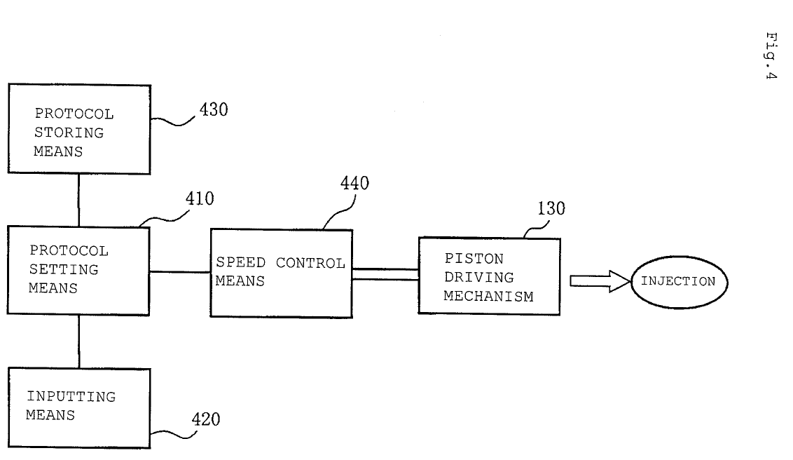

FIG. 3 shows an exemplary protocol of injection performed by the injector according to the present invention. VA represents the injection speed of the contrast medium over time, while VB represents the injection speed of the physiological saline over time. As described before, in the cross injection protocol performed by the injector according to the present invention, injection speed VA of the contrast medium is higher than injection speed VB of the diluent at time T1, the injection speed of the contrast medium is monotonously reduced and the injection speed of the diluent is monotonously increased from time T1 to time T2, and injection speed VB of the diluent exceeds injection speed VA of the contrast medium at time T2.

FIG. 3 shows a preferable embodiment of the cross injection protocol in which the contrast medium is injected at a constant injection speed VA1 from the start of the injection to time T1. On the other hand, the physiological saline is maintained at constant injection speed VB1 until time T1, and in a particularly preferable embodiment, VB1 is equal to 0 ml/sec (that is, no injection is performed). After time T1, the injection speed of the contrast medium is linearly reduced while the injection speed of the physiological saline is linearly increased, and the speed VA and the speed VB cross each other on the way. At time T2, the injection speed of the contrast medium is reduced to VA2, while the injection speed of the physiological saline is increased to VB2. In a particularly preferable embodiment, VA2 is equal to 0 ml (that is, injection is finished). In addition, VA plus VB is preferably constant from T1 to T2. After T2, the injection of the physiological saline may be continued or stopped.

Such a cross injection protocol is preferably used especially for X-ray diagnosis of heart diseases. Specifically, in the examination of an atrial septum or ventricular septum with an angiography apparatus, the contrast medium reaches the right atrium and right ventricle at a different point in time from that when it reaches the left atrium and left ventricle. The contrast medium is injected into the body of a patient through a blood vessel, passes the right atrium and right ventricle, passes the pulmonary artery, lungs, pulmonary vein, and reaches the left atrium and left ventricle through bloodstreams. If only the contrast medium is first injected at a constant speed and the injection of the contrast medium is stopped after a relatively short time period and then switched to injection of physiological saline in accordance with a conventional protocol. In this case only the contrast of right atrium and right ventricle may be increased at early phases, and when the contrast medium passes the lungs and then reaches the left atrium and left ventricle to allow X-ray diagnosis, the contrast medium may have already left from the right atrium and right ventricle, and the effect of the contrast medium may have worn off. On the other hand, if only the contrast medium is injected at a constant injection speed for a longer time period, the contrast medium is mixed with blood during the circulation in the body, so that the concentration of the contrast medium reaching the left atrium and left ventricle earlier may be different from the concentration of the contrast medium reaching the right atrium and right ventricle later, and it causes different contrasts between both sides of the atrial septum or ventricular septum, thereby making it difficult to examine the heart septum.

When the cross injection protocol is used, however, the contrast can be achieved with substantially no difference between both sides of the heart septum. In a typical example, the reduction in injection speed of the contrast medium from VA1 to VA2 is 0.05 to 2 ml/sec/sec, for example, and preferably 0.1 to 1 ml/sec/sec, and more particularly 0.2 to 0.7 ml/sec/sec. On the other hand, the increase in injection speed of the diluent from VB1 to VB2 is 0.05 to 2 ml/sec/sec, for example, and preferably 0.1 to 1 ml/sec/sec, and more particularly 0.2 to 0.7 ml/sec/sec. The following table shows a specific example of the cross injection protocol when a particular contrast medium is used. The example in the table corresponds to the protocol shown in FIG. 3. The injection speed and the injection quantity are determined in view of the characteristics of the patient such as his weight.

TABLE 1

| | T1 | T2 | VA1 | VB1 | VA2 | VB2 |
|---|---|---|---|---|---|---|
| Protocol | (sec) | (sec) | (ml/sec) | (ml/sec) | (ml/sec) | (ml/sec) |
| Example 1 | 10 | 25 | 3 | 0 | 0 | 3 |
| Example 2 | 10 | 20 | 5 | 0 | 0 | 5 |
| Example 3 | 15 | 25 | 4 | 0 | 0 | 4 |

The injector according to the present invention has the structure for performing the cross injection protocol. Specifically, in an embodiment, the injector includes inputting means 420 which receives entry for selection of a protocol and the like as shown in a block diagram of FIG. 4. Inputting means 420 corresponds to the main operation panel 103 provided for injection control unit 101 in FIG. 1. When the cross injection protocol is selected through inputting means 420, protocol setting means 410 refers to protocol storing means 430 in order to call a basic pattern of the cross injection protocol. After a selection or input screen is displayed on display 104, entry is made to specify a set of necessary parameters such as T1, T2, VA1, VB1, VA2, and VB2, for example. Protocol setting means 410 and protocol storing means 430 are contained as a function of injection control unit 101 by using hardware and/or software. The pattern of the cross injection protocol may be directly entered with an inputting device. For example, as described in Japanese Patent Laid-Open No. 2004-298549, the pattern of the cross injection protocol may be entered through drawing on a touch panel with a stylus.

After the pattern of the cross injection pattern is determined in this manner, the pattern is sent as data to speed control means 440. Speed control means 440 is also contained as a function of injection control unit 101 by using hardware and/or software. Alternatively, all or some of the functions thereof may be provided for injection head 110. The speed control means 440 moves two piston driving mechanisms 130 (see FIG. 2) forward so as to perform the cross injection protocol, thereby performing injection.

The syringe information including the capacity of the syringe, particularly the cross-sectional area thereof, has been acquired in advance, for example by making entry with inputting means 420 or by recognizing the syringe information provided for the syringe (for example with an IC chip or the like) with a recognition system provided for injection head 110. Thus, speed control means 440 determines the forward speed of piston driving mechanisms 130 in view of the syringe cross-sectional area.

In the above description, the “means” forming part of the system according to the present invention may be a dedicated mechanism or may double as another means, or may be a logical configuration on a computer system. Those skilled in the art can readily provide the specific configuration thereof by referring to the specification.

### INDUSTRIAL AVAILABILITY

The present invention can provide a chemical liquid injector which can inject a contrast medium in accordance with an optimal injection pattern for a diagnostic image of a particular body section.

1. An injector wherein at least two syringes including a syringe filled with a contrast medium and a syringe filled with a diluent are mounted, and comprises at least two piston driving mechanisms which individually operates the two syringes,
the injector further comprising:
a protocol setting means for setting an injection protocol in which each of injection speeds of the contrast medium and the diluent is represented over time; and
a speed control means for controlling operation of the two piston driving mechanisms in accordance with the set injection protocol,
the protocol setting means can receive an setting of an injection protocol (referred to as a cross injection protocol) in which the injection speed of the contrast medium is higher than the injection speed of the diluent from start of injection to time T1, the injection speed of the contrast medium is monotonously reduced and the injection speed of the diluent is monotonously increased from time T1 to time T2, and the injection speed of the diluent exceeds the injection speed of the contrast medium at time T2.

2. The injector according to claim 1, further comprising a protocol storing means storing the cross injection protocol.

3. The injector according to claim 2, wherein the protocol storing means stores a basic pattern of the cross injection protocol, and the basic pattern is read out in response to read-out operation performed by the protocol setting means.

4. The injector according to claim 1, wherein the injection speed of the contrast medium is constant from the start of injection to time T1 and is linearly reduced from T1 to T2, and
the injection speed of the diluent is constant from the start of injection to time T1 and is linearly increased from T1 to T2.

5. The injector according to claim 4, wherein the injection speed of the diluent is equal to 0 ml/sec from the start of injection to time T1, and
the injection speed of the contrast medium is equal to 0 ml/sec at time T2.

6. The injector according to claim 4, wherein the sum of the injection speed of the contrast medium and the injection speed of the diluent is constant from the start of injection to time T2.

7. The injector according to claim 1, wherein the injector is used in X-ray diagnosis of a heart disease.
